(19) United States
(12) Patent Application Publication
(10) Pub. No.: US 2026/0047796 A1
(43) Pub. Date: Feb. 19, 2026

(54) MULTIMODAL AUTOMATED ACUTE STROKE DETECTION

(71) Applicant: Neuronics Medical Inc., Los Angeles, CA (US)

(72) Inventors: Radoslav Raychev, Los Angeles, CA (US); Todor Todorov, Sofia (BG); Svetlin Penkov, Sofia (BG); Krasimir Stoev, Los Angeles, CA (US); James Shanahan, Los Angeles, CA (US); Daniel Angelov, Sofia (BG)

(21) Appl. No.: 19/101,674

(22) PCT Filed: Aug. 18, 2023
(86) PCT No.: PCT/US2023/072519

§ 371 Date: Feb. 6, 2025

Related U.S. Application Data
(60) Provisional application No. 63/371,824, filed on Aug. 18, 2022.

(51) Int. Cl. A61B 5/00; A61B 5/11

(52) U.S. Cl. CPC A61B 5/4064; A61B 5/0077; A61B 5/1128; A61B 5/4803; A61B 5/7267; A61B 5/7282; A61B 2562/0219

(57) ABSTRACT

A method for stroke detection is provided. A data capture module captures input data from a plurality of sensors, in response to user assessment instructions for a person to look at one or more camera, perform one or more arm exercises, and perform one or more speech acts. A perception module generates summaries of the input data corresponding to artifacts associated with one or more machine learning models. A classification module accepts as input the input data from the data capture module and the summaries from the perception module. Based on the input data and the summaries, a classification module assigns a stroke classification label and a corresponding probability. The classification module outputs a recommendation according to the stroke classification label and the corresponding probability.

### CROSS-REFERENCE TO RELATED APPLICATION(S)

This application claims the benefit of U.S. Provisional Patent Application No. 63/371,824, filed Aug. 18, 2022, which is hereby incorporated by reference herein in its entirety.

### BACKGROUND

A stroke refers to a sudden interruption of blood supply to the brain, leading to the loss of brain function. It can be caused by a blockage in a blood vessel (ischemic stroke) or by the rupture of a blood vessel (hemorrhagic stroke). Strokes can have severe consequences, including physical impairments, cognitive deficits, and even death. The symptoms of a stroke can vary depending on the specific type of stroke (ischemic or hemorrhagic) and the area of the brain affected. Common symptoms of a stroke include, for example: sudden numbness or weakness in the face, arm, or leg, typically on one side of the body: trouble speaking or understanding speech: confusion or difficulty comprehending simple instructions: trouble seeing in one or both eyes, such as blurry vision or loss of vision; sudden severe headache with no known cause; trouble with coordination, dizziness, or loss of balance; and/or difficulty walking or a sudden loss of balance or coordination. Such symptoms can appear suddenly and without warning.

A stroke may be reversible if caught and treated early. However, less than 5% of all acute stroke patients are treated in the “golden” three hour time window due to delays in diagnosis and poor stroke recognition among caregivers, patients, and families.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an overview of an example process flow for automating a FAST protocol for detection of acute stroke according to certain embodiments.

FIG. 2 illustrates a modular overview of an example process flow for automating the FAST protocol for detection of acute stroke according to certain embodiments.

FIG. 3 illustrates an example processing flow of a pipeline for processing facial videos according to one embodiment.

FIG. 4A, FIG. 4B, FIG. 4C, FIG. 4D, FIG. 4E, FIG. 4F, FIG. 4G, and FIG. 4H are annotated images of a patient's face used to define different classes of facial landmarks according to one embodiment.

FIG. 5 illustrates an example processing flow of a pipeline for detecting arm weakness by analyzing various motion specific metrics according to one embodiment.

FIG. 6 illustrates filtered and normalized acceleration signals, angular velocity signals, and magnetic field signals processed according to certain embodiments.

FIG. 7A illustrates example acceleration signals processed according to certain embodiments.

FIG. 7B illustrates example angular velocity signals processed according to certain embodiments.

FIG. 8A illustrates example acceleration signals and angular velocity signals processed according to certain embodiments for an arm of a healthy person.

FIG. 8B illustrates example acceleration signals and angular velocity signals processed according to certain embodiments for an arm with subtle weakness.

FIG. 8C illustrates example acceleration signals and angular velocity signals processed according to certain embodiments described for an arm with moderate weakness.

FIG. 9 illustrates an example processing flow of an audio processing pipeline according to one embodiment.

FIG. 10 illustrates an example of a FAST AI online inference pipeline wherein a current video and baseline video may be compared against each other according to one embodiment.

FIG. 11 illustrates a flowchart of a method for stroke detection, according to embodiments herein.

FIG. 12 is a schematic illustration of a computing system arranged in accordance with examples of the present disclosure.

### DETAILED DESCRIPTION

### Approach Overview

Embodiments disclosed herein provide an artificial intelligence (AI)-enabled automated solution for clinical diagnosis of stroke. Such embodiments may help increase stroke treatment by improving acute recognition and diagnosis.

Certain embodiments use the FAST (Face, Arm, Speech, Time to call 911) and/or BE FAST (Balance, Eyes, Face, Arms, Speech, Time to call 911) paradigms for acute stroke recognition. The FAST and/or BE FAST paradigms may also be referred to herein approaches or protocols. The FAST approach is a simple and effective method for quickly identifying the signs of a stroke. The FAST approach includes looking for face drooping, which may include unevenness or drooping on one side of the face. A user of the approach (e.g., medical personnel, a family member, or a friend) may, for example, ask the person to smile and observe if one side of the face does not move as well as the other. The FAST approach further checks for arm weakness. For example, the user may ask the person to raise both arms. If one arm drifts downward or cannot be held up compared to the other, it may indicate arm weakness. The FAST approach further checks for speech difficulties, wherein the user listens carefully to the person's speech. Slurred speech, difficulty in finding words, or the person being unable to speak or understand speech are potential signs of a stroke.

Certain embodiments disclosed herein use the FAST approach in stroke detection system, such as an automated application executed by a smart phone, for detection of acute stroke signs using machine learning (ML) algorithms for recognition of facial asymmetry, arm weakness, and speech changes. The ML algorithms may also base detection of the stroke on other characteristics such as balance or eye movements (e.g., gaze). If the stroke detection system detects or predicts that a person has any of symptoms (e.g., facial asymmetry, arm weakness, slurred speech, imbalance, abnormal gaze movements), the stroke detection system may automatically call emergency services. To enable automatic assessment of the core FAST components, certain embodiments may use multi-modality machine learning methods that may be designed with particular tasks in mind.

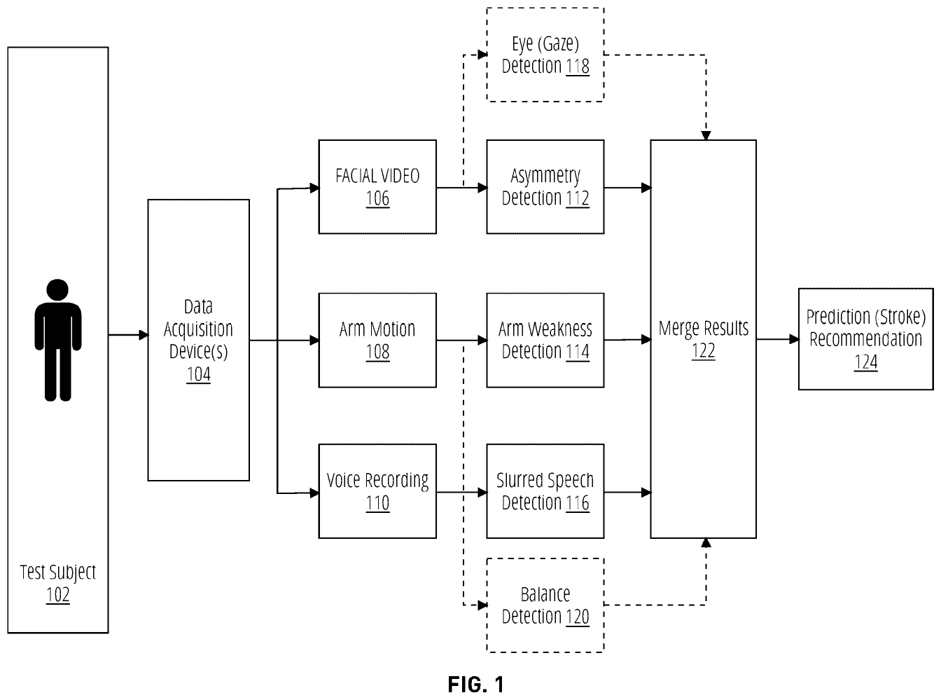

At a high level, FIG. 1 illustrates an overview of an example process flow for automating a FAST protocol for detection of acute stroke according to certain embodiments. A test subject 102 may interface with a data acquisition device or data acquisition devices 104. The test subject 102 may also be referred to as a subject, a person, or a patient. The data acquisition devices 104 may collect various types of data. For example, the data acquisition devices 104 may collect facial video data 106 of the test subject 102, arm motion data 108 corresponding to one or more arm motion measurements of the test subject 102, and/or voice recording data 110 corresponding to speech by the test subject 102. These three data modalities may be processed independently and then merged together to generate a diagnosis of a stroke.

As shown in FIG. 1, the automation of the FAST protocol may be achieved by independently processing three or more data modalities used for the assessment of the test subject 102. For example, the facial video data 106 may be processed for asymmetry detection 112, wherein the test subject 102 is asked to perform certain facial movements (e.g., as prescribed by the FAST protocol) while a video of their face is being recorded. The arm motion data 108 may be processed for arm weakness detection 114, wherein the test subject 102 is asked to raise and keep their hands in a particular position (e.g., as prescribed by the FAST protocol) while they hold a device capable of recording acceleration, rate of rotation and strength of the ambient magnetic field in three dimensions. In other embodiments, the motion may be determined from video data. The voice recording data 110 may be processed for slurred speech detection 116, wherein the test subject 102 is asked to read aloud several worlds (e.g., as prescribed by the FAST protocol) while high quality audio is being recorded. In addition, or in other embodiments, the facial video data 106 may be processed for eye (gaze) detection 118 and/or the arm motion data 108 or other motion data may be processed for balance detection 120.

The information used for the data modalities may be gathered during a self-assessment performed using the stroke detection system by the test subject 102 themselves or by a third party, such as a paramedic or triaging personnel.

In order for the embodiments to be as flexible as possible with respect to the hardware device(s) used for the acquisition of the data, each data modality may be processed independently of the others and the results may be merged 122 to generate an output 124 including a prediction (e.g., of a stroke) or recommendation (e.g., to seek emergency medical treatment). This may enable a much more extensive analysis of the performance of the underlying machine learning models that can be performed over each of the available data modalities independently.

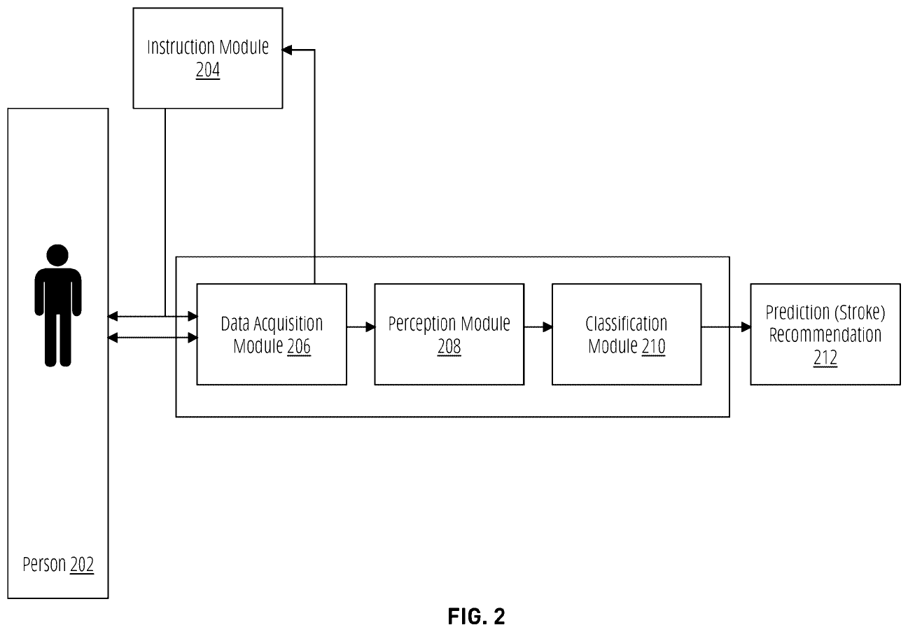

FIG. 2 illustrates a modular overview of an example process flow for automating the FAST protocol for detection of acute stroke according to certain embodiments. An instruction module 204 may instruct a person 202 who is or may be experiencing a stroke, or may have experienced a stroke in the past, in a sequential or parallel manner to look at a device (e.g., a camera or a camera of a mobile phone), perform arm exercises, and perform some speech acts. A data acquisition module 206 captures data about the person 202 from various sensors such as a color camera (e.g., a red-green-blue (RGB) or an RGB-depth (RGBD) camera), an audio capture device, and motion sensors such as an accelerometer, magnetometer, and/or gyroscope. A perception module 208 may summarize the captured data into high-level artifacts such as pose or location points for a face, an arm motion, and speech that is summarized as Mel Frequency Cepstral Coefficients (MFCC). A classification module 210 accepts as input the raw sensor data and the summaries from the perception module 208, and may assign a stroke classification label and a corresponding probability. The data acquired by the data acquisition module 206 may include video of the person 202, arm motion measurements, and/or voice recording. These three data modalities may be processed independently and then merged together in order to generate a diagnosis of stroke. An output 212 may include a prediction (e.g., stroke) and/or a recommendation (e.g., to seek emergency medical treatment).

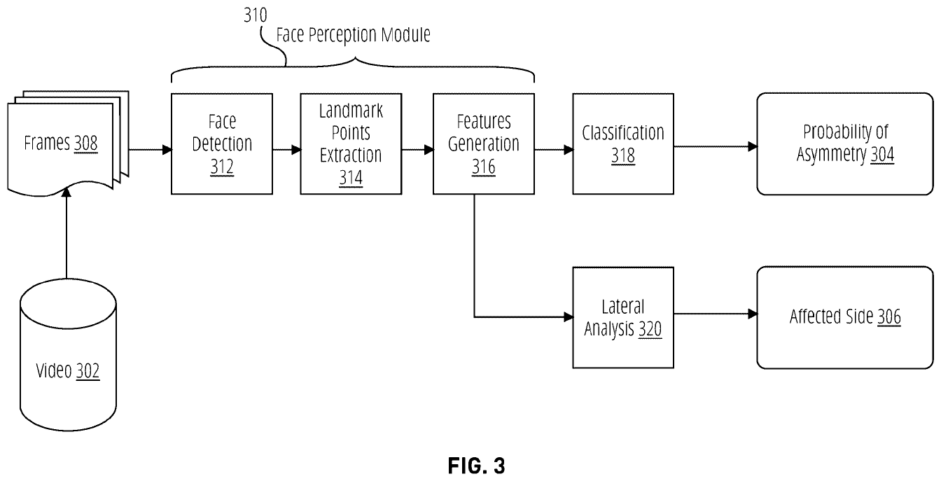

FIG. 3 illustrates an example processing flow of a pipeline for processing facial videos according to one embodiment. For a single test subject video 302, the output of the pipeline may include an estimated probability 304 of facial asymmetry being present, an estimated uncertainty (not shown) of the prediction, and an indication of an affected side 306 of the face if asymmetry is present.

### Detecting Facial Asymmetry

The pipeline for detecting facial asymmetry may perform multiple processing steps, as illustrated in FIG. 3, to make a prediction if facial asymmetry is present in the video 302. In certain embodiments, the perception module 208 shown in FIG. 2 includes a face perception module 310, as shown in the pipeline of FIG. 3. The face perception module 310 includes a face perception module 310 for face detection, a facial landmark detector 314 for landmark points extraction, and a features generator 316 for features generation.

The processing flow starts by taking in a video V (shown as video 302) that is split into frames F_{1, . . . , N} (shown as frames 308). Each frame Fᵢ may then processed by the face detector 312 that outputs bounding boxes b_{1, . . . , M}_{ᵢ}, where Mᵢ is the number of faces detected in frame Fᵢ. The largest detected face in a frame may be found by applying non-maximal suppression based on the bounding box area such that Bᵢ=argmax({area(bᵢ)|bᵢ∈b_{1, . . . , M}_{ᵢ}}). As a result, there may be N bounding boxes denoted as B_{1, . . . , N}. Each bounding box is then passed through the facial landmark detector 314 resulting in a set Lᵢ={l_{i,1}, i_{i,2}, . . . , l_{i,K}} where l_{i,j}∈[0; 1]² is a two dimensional (2D) location with normalized coordinates between 0 and 1 with respect to Bᵢ and K is the number of detected facial landmark points in frame Fᵢ.

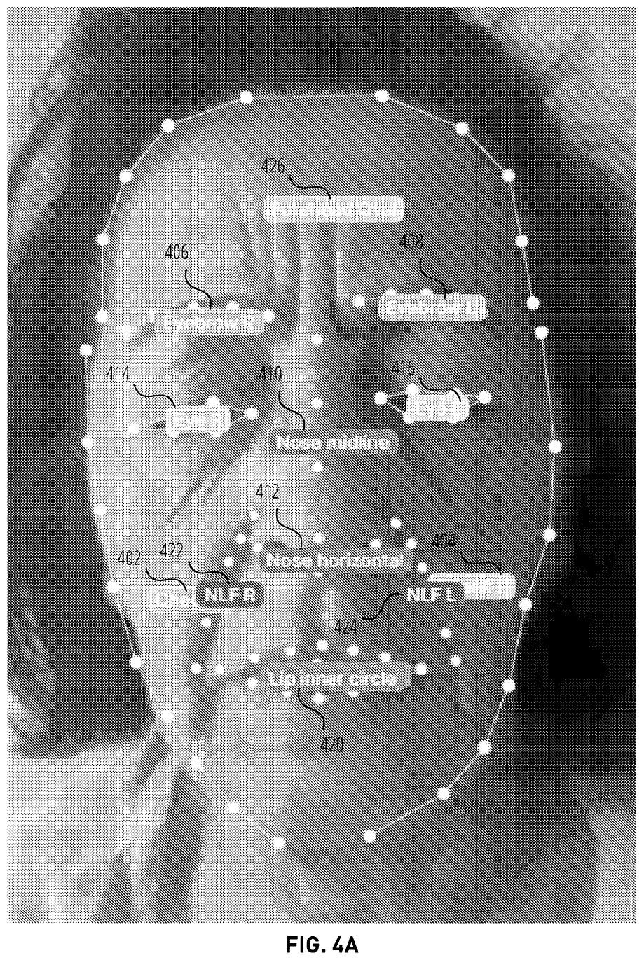

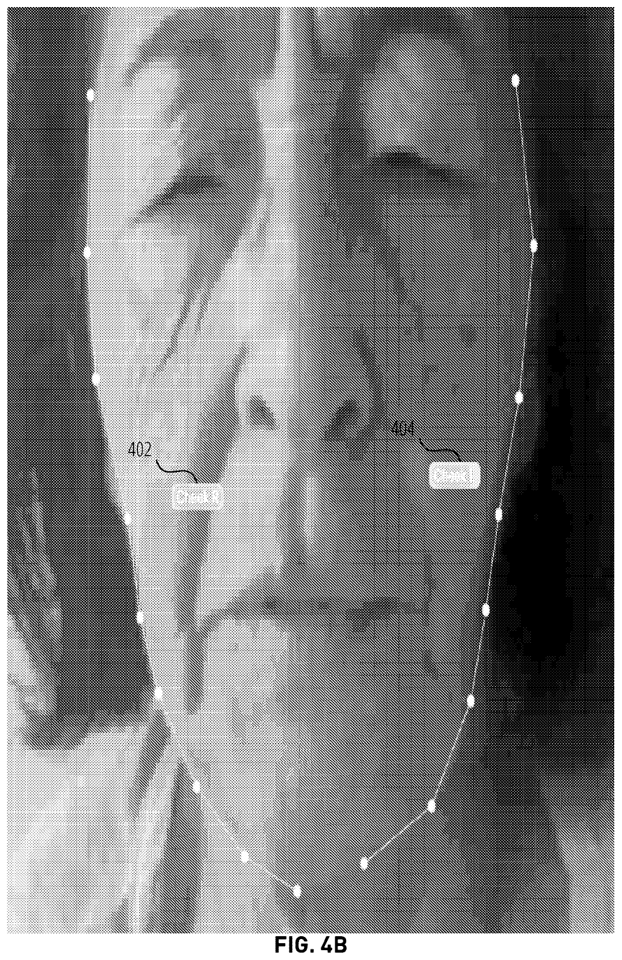

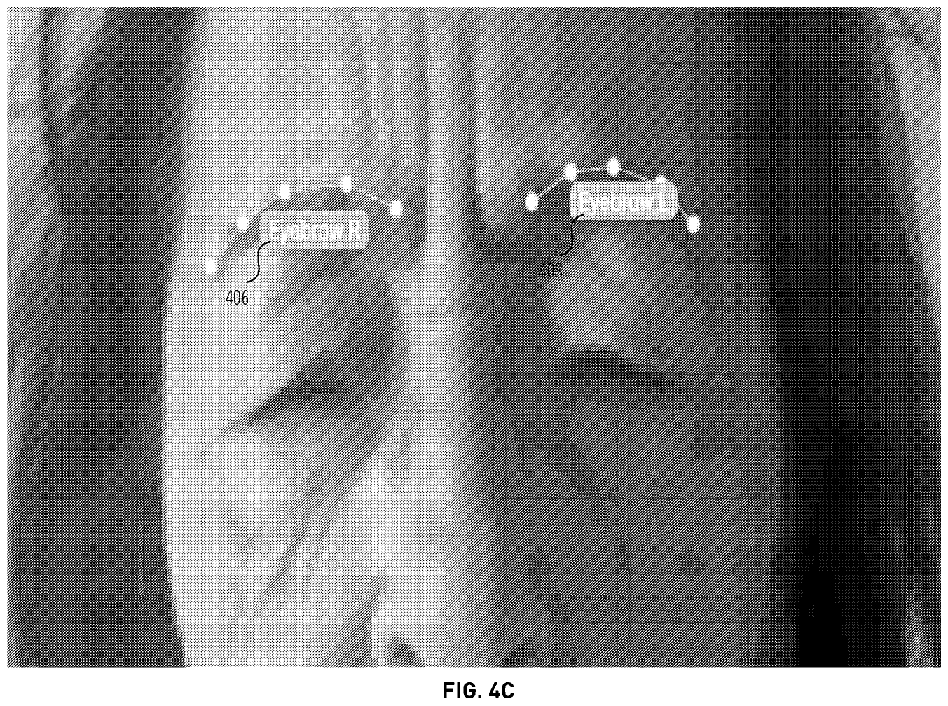

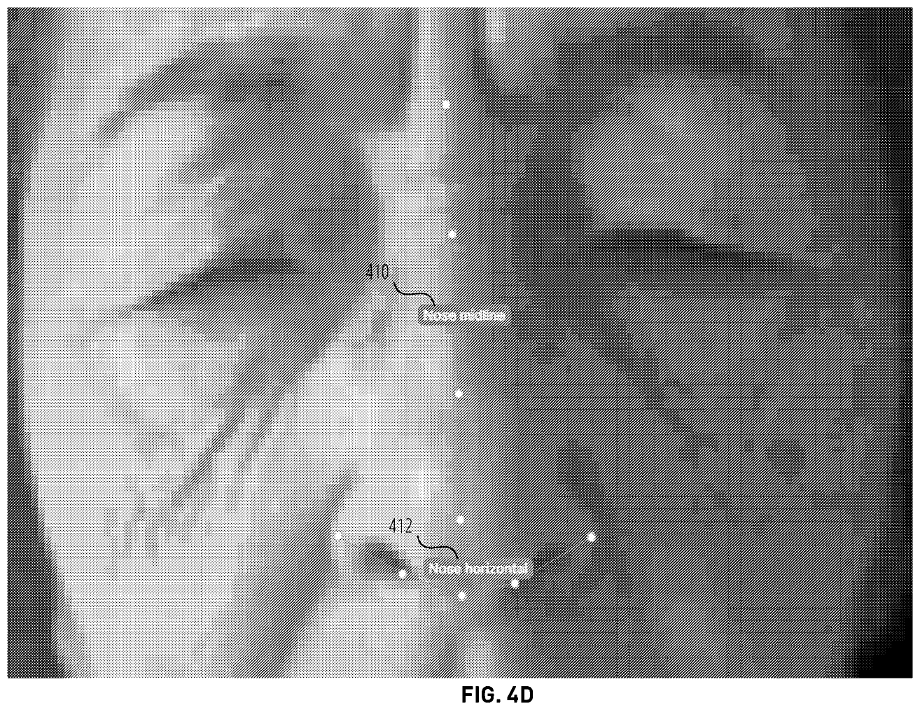

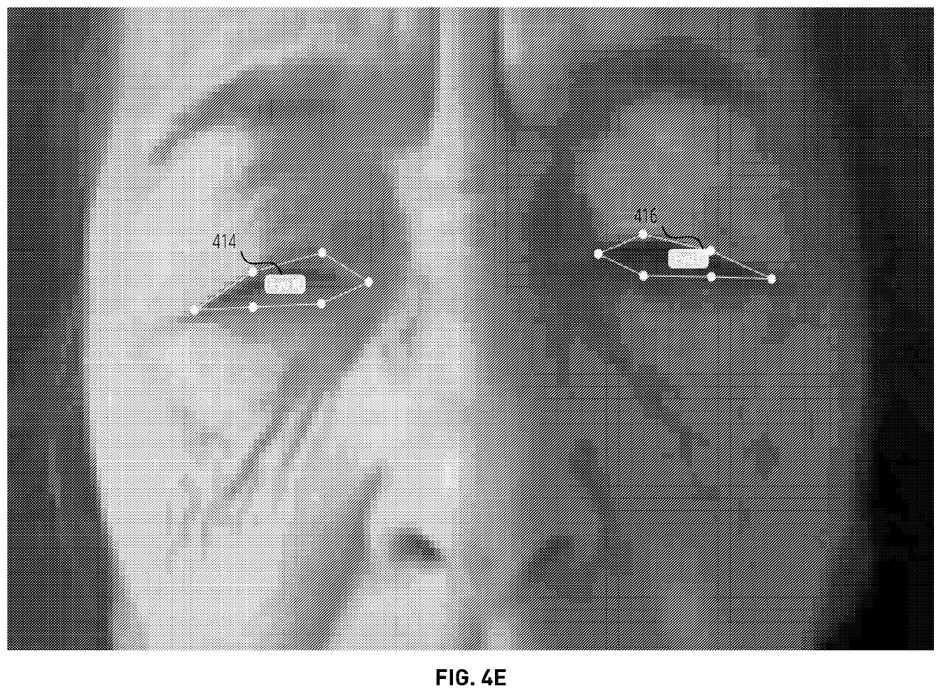

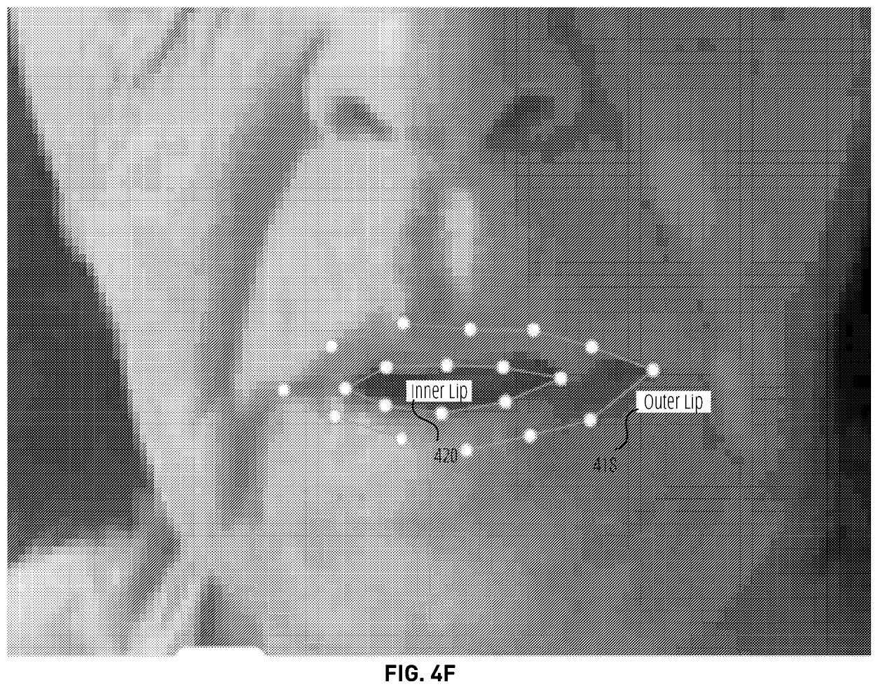

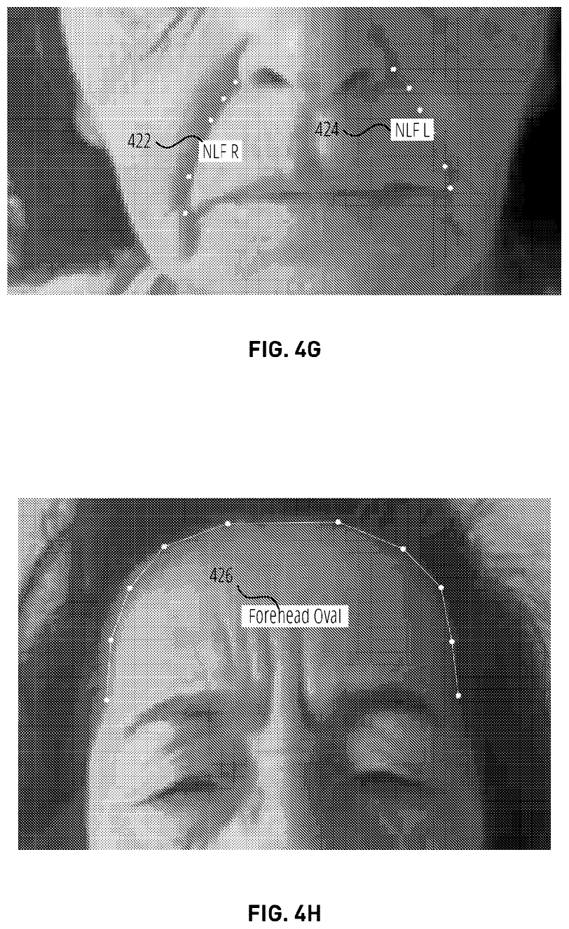

In some embodiments, the facial landmark detector 314 may be trained to extract a standard 68 key points that are widely used by the machine learning community. See, for example, Hohman. Marc H., et al. “Determining the threshold for asymmetry detection in facial expressions.” The Laryngoscope 124.4 (2014): 860-865. In other embodiments, however, the facial landmark detector 314 may be trained on a custom set of facial landmark points that has been identified by stroke specialists. For example, as discussed herein with respect to FIG. 4A to FIG. 4H, certain embodiments use at least 90 location points to define facial landmarks for stroke detection.

The features generator 316 is configured to determine a set of facial feature vectors from the facial landmarks for each of the sequence of video frames. In some cases, directly processing the coordinates of the detected landmark points may yield a classifier with poor generalization capabilities as it may be sensitive to the location and orientation of the face in the image. To reduce or avoid these issues, the facial landmark points may be converted into a set of distances Dᵢ={L₂(l_{i,j}, l_{i,k})|l_{i,j}, l_{i,k}∈Lᵢ, i≠j} with cardinality

$$\vert {D}_{i} \vert = \frac{K * ( K - 1 )}{2}$$

and then a dimensionality reduction may be performed using principal components analysis (PCA) to obtain a final feature vector xᵢ^{face}∈ [symbol] for every video frame Fᵢ, where q is the target dimensionality for the PCA step. In some example embodiments, q=100 may be sufficient to explain more than 99% of the variance in Dᵢ for K=68.

The classification module 318, which may include or may be referred to as a facial asymmetry submodule, determines a presence of facial asymmetry based on the set of facial feature vectors. To do so, the classification module 318 may use a classifier f_{asymmetry} that takes as an input xᵢ^{face} and outputs

$${\hat{y}}_{i}^{face} = p ( {y}_{asymmetry} = 1 \vert x = {x}_{i}^{face} ) = {f}_{asymmetry} ( {x}_{i}^{face} ) ,$$

where y_{asymmetry}∈{0, 1} may indicate the presence of facial asymmetry. After extensive model comparison, the inventors of the present application determined that a linear discriminant analysis (LDA) is well suited for this classification task.

Processing every frame Fᵢ, i∈{1, . . . , N} in the video may result in N predictions

$${\hat{y}}_{1 , … , N}^{face}$$

that may be aggregated using a kernel density estimation (KDE) to determine a mean predicted probability of asymmetry as well as an uncertainty of the estimate.

In addition, certain embodiments include a lateral analysis submodule 320 to perform a lateral analysis of observed face movements to identify which side of the face is likely affected. The analysis may be based on measuring the total movement of the left and right sides of the face and determining which side has moved less throughout the observed video. In particular, the set of normalized facial landmark points Lᵢ={l_{i,1}, i_{i,2}, . . . , l_{i,K}} may be split into subsets L_{i, left} and L_{i, right} including the facial landmark points that belong to the left and right side of the face, respectively, detected at video frame Fᵢ. Any points along the central vertical line of the face are included in both sets. The total displacement of facial landmark points on each side of the face may be estimated as

$${d}_{i , left} = \sum \text{?} {L}_{2} ( \text{?} {e}_{i , left} ) and$$

$${d}_{i , left} = \sum \text{?} {L}_{2} ( \text{?} {e}_{i , right} ) ,$$

$$\text{?} \text{indicates text missing or illegible when filed}$$

where ε_{i, left} and ε_{i, right} denote the locations of the center of the left eye and the right eye, respectively, in frame Fᵢ, and L₂ denotes the Euclidean norm. Processing the sequence of video frames results in the sequences d_{1, left}, d_{2, left}, . . . , d_{N, left} and d_{i, right}, d_{2, right}, . . . , d_{N, right} whose variances σ²_{left} and σ²_{right} indicate how much the left and right side of the face has moved throughout the video. The side with the lower variance is predicted to be the affected side 306.

Thus, the pipeline shown in FIG. 3 automates the detection of facial asymmetry, which is one of the symptoms assessed by the FAST protocol.

As discussed above, in some embodiments, the facial landmark detector 314 may be trained to extract at least 90 points to identify, define, or track facial landmarks. For example, FIG. 4A, FIG. 4B, FIG. 4C, FIG. 4D, FIG. 4E, FIG. 4F, FIG. 4G, and FIG. 4H are annotated images of a patient's face wherein 90 location points are used to define thirteen different classes of facial landmarks according to one embodiment. The annotations and facial landmarks are used to determine facial asymmetry in a video input of the patient while they talking and/or making facial expressions. Groups of the location points are connected and form a curve or shape corresponding to a respective part of the face.

In this example, the annotations include Cheek R 402 and Cheek L 404, which are intentionally partially covered in FIG. 4A and shown in FIG. 4B. The annotation Cheek R 402 corresponds to the right cheek and includes nine points placed on the right side of the face (from the patient's point of view). The first point may begin from the upper end of the right ear (if the right ear is visible) or from the lower end of the right eyebrow (if the right ear is not visible). The location points may follow the contour of the face down to the bottom edge of the chin and may be distributed as evenly as possible.

The annotation Cheek L 404 corresponds to the left cheek and includes eight points that may be placed on the left side of the face (from patient's point of view). The first point may begin from the left edge of the chin, symmetrical to the second to last point from the Cheek R 402. Each location point may follow the contour of the face up to the upper end of the left ear (if the left ear is visible) or to the lower end of the left eyebrow (if the left ear is not visible).

In this example, the annotations also include Eyebrow R 406 and Eyebrow L 408 shown in FIG. 4A and FIG. 4C. The annotation Eyebrow R 406 includes five points that may be placed on the right eyebrow (from the patient's point of view). The location points may start from the outer corner and end on the inner corner of the right eyebrow. The location points may follow the upper contour of the right eyebrow and may be distributed as evenly as possible.

The annotation Eyebrow L 408 includes five points that may be placed on the left eyebrow (from the patient's point of view). The location points may start from the inner corner and end on the outer corner of the left eyebrow. The location points may follow the upper contour of the left eyebrow and may be distributed as evenly as possible.

In this example, the annotations also include Nose midline 410 and Nose horizontal 412 shown in FIG. 4A and FIG. 4D. The annotation Nose midline 410 includes four points that may start from the center between the eyebrows and end on the tip of the nose. The other location points may follow the front contour of the nose and may be distributed as evenly as possible.

The Nose horizontal 412 includes five points that may begin with a first point on the right outer tip of the right nostril (from the patient's point of view). A second point may be on the inner edge of the right nostril. A third point may be between the two nostrils. A fourth point may be on the inner edge of the left nostril. A last point may be on the outer tip of the left nostril.

In this example, the annotations also include Eye R 414 and Eye L 416 shown in FIG. 4A and FIG. 4E. The Eye R 414 includes six points placed on the right eye (from the patient's point of view). A first point may be placed on the outer edge of the right eye. The next point may be placed on the inner edge of the right eye. The location points may be associated with identifiers (IDs) and be placed clockwise. The other four points may be placed on the outer contours of the right eye so that a first pair of points are aligned vertically and a second pair of points are aligned vertically. If the right eye is completely shut, then the first pair of points may at least partially overlap and the second pair of points may at least partially overlap.

The Eye L 416 includes six points placed on the left eye (from the patient's point of view). A first point may be placed on the inner edge of the left eye. The next point may be placed on the outer edge of the left eye. The location points may be associated with IDs and be placed clockwise. The other four points may be placed on the outer contours of the left eye so that a first pair of points are aligned vertically and a second pair of points are aligned vertically. If the left eye is completely shut, then the first pair of points may at least partially overlap and the second pair of points may at least partially overlap.

In this example, the annotations also include Outer Lip 418 and Inner Lip 420 shown in FIG. 4F. In FIG. 4A, Outer Lip 418 is intentionally covered (although many of the corresponding location points are shown) and Inner Lip 420 is shown as “Lip inner circle” (with many of the corresponding location points being covered).

The Outer Lip 418 includes twelve points placed on the outer contours of the mouth of the patient. A first point may be placed on the right edge of the lips (from the patient's point of view). A second point may be placed on the left edge of the lips. The rest of the points may follow the outer contour and are arranged such that each point on the upper lip may be vertically aligned to each point on the bottom lip.

The Inner Lip 420 includes eight points that may be placed on the inner contours of the lips of the patient. A first point may be placed on the right edge of the inner contour (from the patient's point of view). A second point may be placed on the left edge. The rest of the points may follow the inner contour of the lips as they follow an open mouth. Each upper point may be vertically aligned to each lower point. The points may be evenly distributed along the lips edges. The corresponding points may at least partially coincide when the mouth is shut.

In this example, the annotations also include NLF R 422 and NLF L 424 shown in FIG. 4A and FIG. 4G. The NLF R 422 includes six points that may be placed along patient's nasolabial fold (NLF) on the right side of the face (from the patient's point of view). The points may start from the right outer edge of the nose and may be distributed evenly down the NLF to the right outer edge of the mouth.

The NLF L 424 includes six points that may be placed on the left side of the face (from the patient's point of view). The points may start from the left outer edge of the nose and may be distributed evenly down the NLF to the left outer edge of the mouth.

In this example, the annotations also include Forehead Oval 426 shown in FIG. 4A and FIG. 4H. The Forehead Oval 426 includes ten points that may be placed on the forehead of the patient and may follow the outer contours of the head and the hairline of the forehead. A first point may be placed on the right temple (from the patient's point of view). A second point may be placed on the left temple (from the patient's point of view). The rest of the points may follow the hairline.

Detecting Arm Weakness from Motion Data

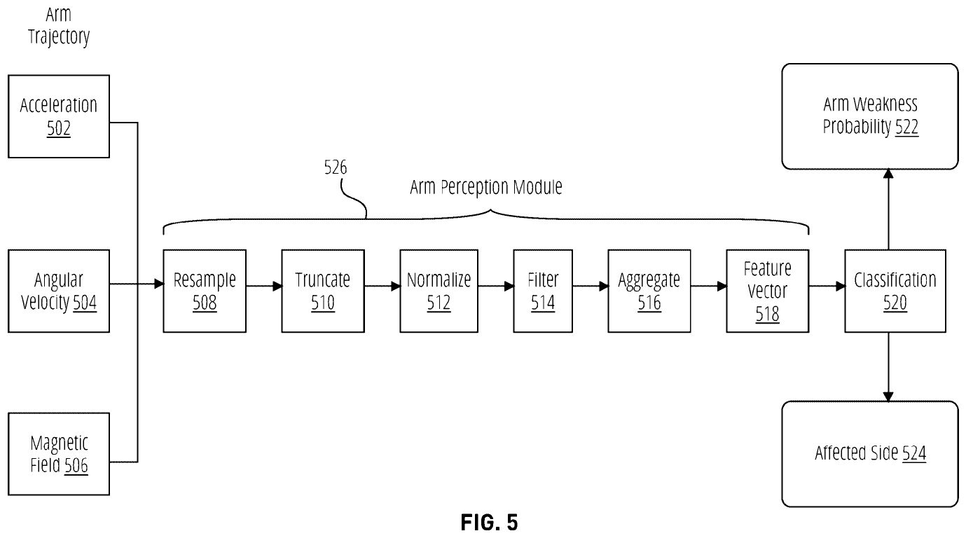

FIG. 5 illustrates an example processing flow of a pipeline for detecting arm weakness by analyzing various motion specific metrics according to one embodiment. In certain embodiments, for example, the subject may hold a device that may record any, or all, of the input motion signals. In other embodiments, video from one or more cameras may be processed to obtain the input motion signals.

The input motion signals may be processed through multiple stages to predict the probability of arm weakness. Also, by comparing predictions made for the left and right arm, the affected side may also be identified.

In some embodiments, detecting arm weakness may be a symptom assessed by the FAST protocol. As prescribed by the FAST protocol, the test subject may be asked to steadily raise their hands sideways or forward and keep that position for several seconds. In this example, the disclosed method for arm weakness detection assumes that the patient holds in their hand, or alternatively wears on their hand or arm, a one or more devices that may be capable or capturing one or more signals including: a three dimensional (3D) acceleration signal 502 denoted as

$${x}_{1 , … , {N}_{accel}}^{accel}$$

where

$${x}_{i}^{accel}$$

∈ [symbol] and N_{accel} is the number of acceleration measurements; a 3D angular velocity signal 504 denoted as

$${x}_{1 , … , {N}_{gyro}}^{gyro}$$

where

$${x}_{i}^{gyro}$$

∈ [symbol] and N_{gyro} is the number of angular velocity measurements; and a 3D magnetic field direction signal 506 denoted as

$${x}_{1 , … , {N}_{magnet}}^{magent}$$

where

$${x}_{i}^{magnet}$$

∈ [symbol] ³ and N_{magnet} is the number of magnetic field measurements.

In certain embodiments, the perception module 208 shown in FIG. 2 includes an arm perception module 526, as shown in the pipeline of FIG. 5. As discussed below, the arm perception module 526 is configured to resample 508, truncate 510, normalize 512, filter 514, aggregate 516, and generate a feature vector 518 from the acceleration signal 502, the angular velocity signal 504, and the magnetic field direction signal 506.

In general, N_{accel}≠N_{gyro}≠N_{magnet} as these signals may be sampled with different frequencies throughout a duration T of the arm weakness test. Therefore, a first step of the arm data processing pipeline may be for the arm perception module 526 to resample 508 the signals to a fixed frequency

$${f}_{sample}^{arm} ,$$

which may result in

$$\hat{N} = T {f}_{sample}^{arm}$$

samples for each of the signal modalities resulting in a resampled signals

$${\hat{x}}_{1 , … , \hat{N}}^{accel} , {\hat{x}}_{1 , … , \hat{N}}^{gyro} , {\hat{x}}_{1 , … , \hat{N}}^{magnet}$$

that have equal sampling frequency and length. The resampling procedure may be performed via piecewise linear interpolation. Furthermore, it may be beneficial to truncate 510 the resampled signals by dropping a small number of samples at the beginning and the end of the test in order to filter out any transitionary artifacts.

In some embodiments, a challenge may be that a person may hold the sensor device with various grasps and in different orientations. Therefore, in certain such embodiments, a z-score is used to normalize 512 the magnitude of each 3D measurement resulting in

$${s}_{1 , … , \hat{N}}^{accel} , {s}_{1 , … , \hat{N}}^{gyro} , {s}_{1 , … , \hat{N}}^{magnet}$$

where

$${s}_{i}^{M} = {L}_{2} ( {x}_{i}^{M} )$$

with M∈{accel, gyro, magnet}, and L₂ denotes the Euclidean norm. Z-score normalization may be further applied resulting in

$${\hat{s}}_{i}^{M} = \frac{{s}_{i}^{M} - {μ}^{M}}{{σ}^{M}} ,$$

where

$${μ}^{M} = E [ {s}_{1 , … , \hat{N}}^{M} ]$$

and variance

$${σ}^{M} = Var ( {s}_{1 , … , \hat{N}}^{M} ) .$$

Additionally, the arm perception module 526 may filter 514 the normalized signals, e.g., using a Butterworth low pass filter with cutoff frequency

$${f}_{cutoff}^{arm} ,$$

to remove high frequency noise artifacts. Then, the arm perception module 526 may aggregate 516 the normalized 512 and filtered 514 signals and generate a single feature vector 518 by concatenation, which results in x^{arm}∈ [symbol] . The test may be performed for both arms results in

$${x}_{left}^{arm} , {x}_{right}^{arm}$$

∈ [symbol] .

The pipeline for detecting arm weakness shown in FIG. 5 includes a classification module 520 to evaluate whether arm weakness is present or not. The classification module 520 outputs an arm weakness probability 522 and an indication of an affected side 524. The classification module 520 may use a classifier f_{weakness} takes as an input x^{arm} and outputs [symbol] =p( [symbol] _{weakness}=1|x=x^{arm})=f_{weakness}(x^{arm}), where [symbol] _{weakness}∈{0, 1} indicates the probability of a presence of arm weakness. After extensive model comparison, the inventors of the present application determined that logistic regression (LR) is well suited for this classification task. If the output of the classifier for either of the arms is positive then arm weakness may be predicted to be present.

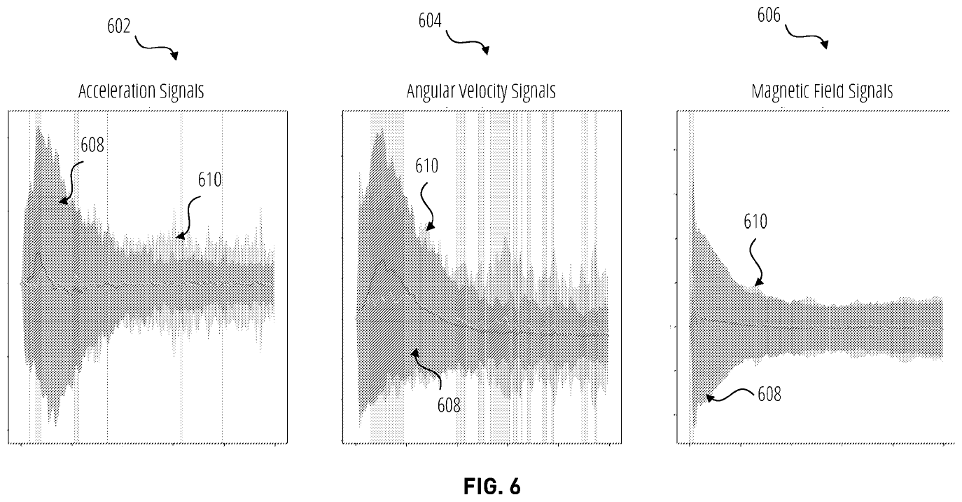

By way of example, FIG. 6 illustrates filtered and normalized acceleration signals 602, angular velocity signals 604, and magnetic field signals 606 processed according to certain embodiments described with respect to FIG. 5. Signals 608 are from healthy patients (shown in a relatively darker gray) and signals 610 are from stroke affected patients (shown in a relatively lighter gray), with solid lines representing a mean trajectory and the relatively darker gray or lighter gray regions around the solid lines representing 1σ uncertainty ranges.

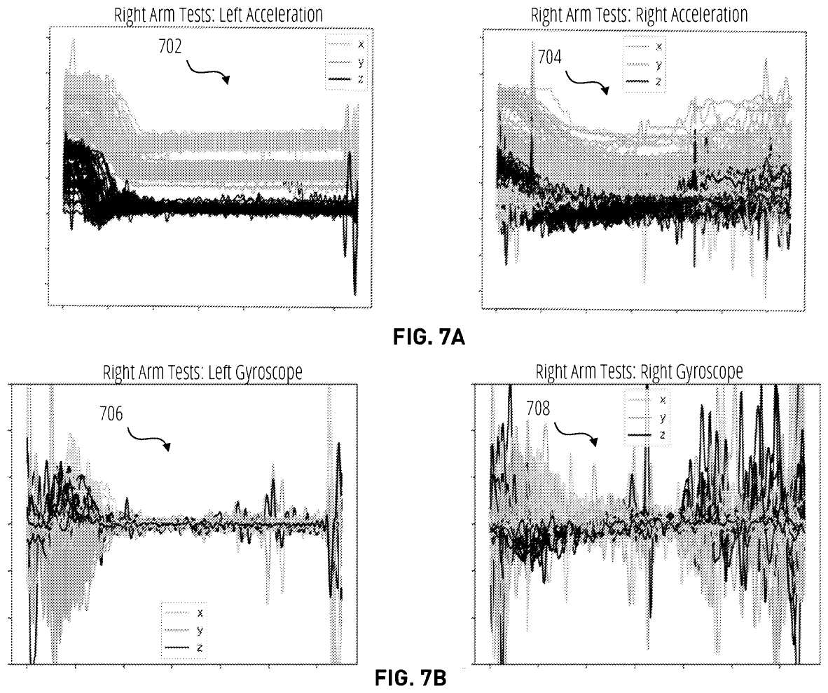

FIG. 7A illustrates example acceleration signals processed according to certain embodiments described with respect to FIG. 5. The acceleration signals were acquired using an accelerometer for a right arm of a person affected by stroke. The acceleration signals 702 correspond to left acceleration of the right arm in an x-axis, a y-axis, and a z-axis. The acceleration signals 704 correspond to right acceleration of the right arm in the x-axis, the y-axis, and the z-axis. The acceleration signals 704 show more variance than the acceleration signals 702, which may indicate arm weakness affected by stroke.

FIG. 7B illustrates example angular velocity signals processed according to certain embodiments described with respect to FIG. 5. The angular velocity signals were acquired using a gyroscope for a right arm of a person affected by stroke. The angular velocity signals 706 correspond to left rotation of the right arm in an x-axis, a y-axis, and a z-axis. The angular velocity signals 708 correspond to right rotation of the right arm in the x-axis, the y-axis, and the z-axis. The angular velocity signals 708 show more variance than the angular velocity signals 706, which may indicate arm weakness affected by stroke.

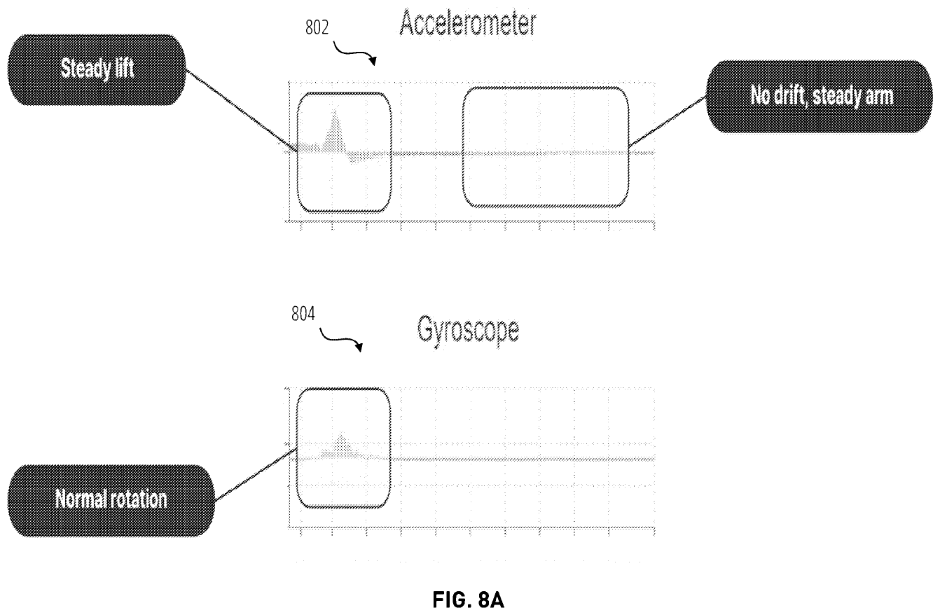

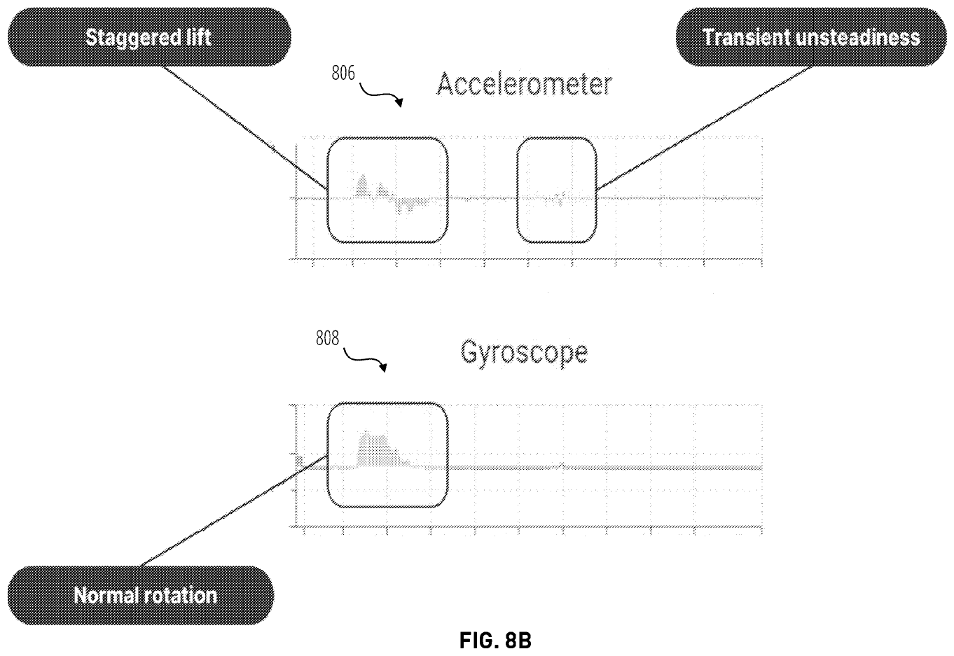

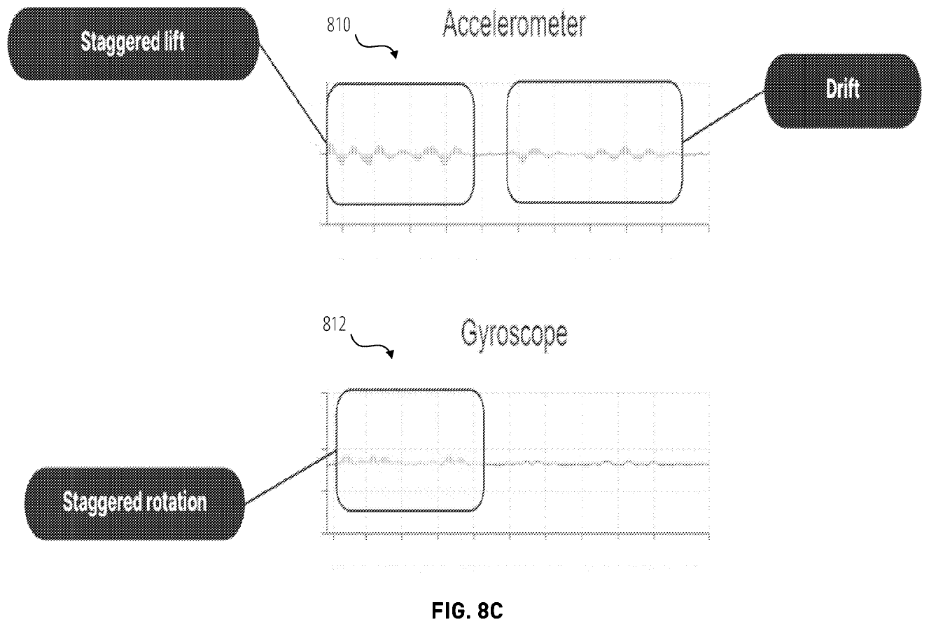

FIG. 8A illustrates example acceleration signals 802 and angular velocity signals 804 processed according to certain embodiments described with respect to FIG. 5 for an arm of a healthy person. The acceleration signals 802 were measured with an accelerometer and show an area of steady lift and an area of no drift indicating a steady arm. The angular velocity signals 804 were measured with a gyroscope and show an area of normal rotation.

FIG. 8B illustrates example acceleration signals 806 and angular velocity signals 808 processed according to certain embodiments described with respect to FIG. 5 for an arm with subtle weakness. The acceleration signals 806 were measured with an accelerometer and show an area of staggered lift and an area of transient unsteadiness. The angular velocity signals 808 were measured with a gyroscope and show an area of normal rotation. The indicated subtle weakness may or may not be a sign of stroke, but may contribute to a prediction of stroke when combined with the other tests of the FAST protocol.

FIG. 8C illustrates example acceleration signals 810 and angular velocity signals 812 processed according to certain embodiments described with respect to FIG. 5 for an arm with moderate weakness. The acceleration signals 810 show an area of staggered lift and an area of drift. The angular velocity signals 812 show an area of staggered rotation. The indicated moderate weakness may lead to a prediction of stroke.

### Detecting Slurred Speech

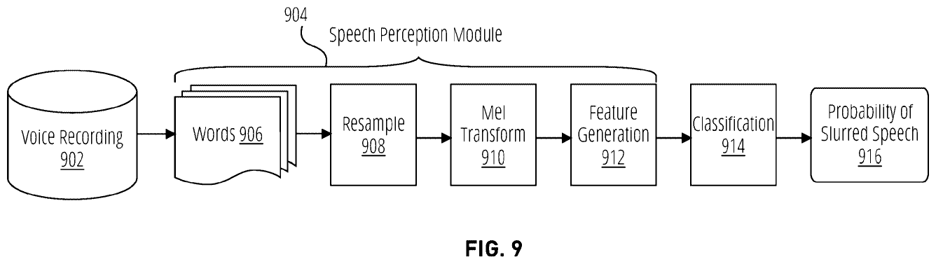

FIG. 9 illustrates an example processing flow of an audio processing pipeline according to one embodiment. In certain embodiments, for example, a voice recording 902 is generated of a subject reading individual words aloud.

In certain embodiments, the perception module 208 shown in FIG. 2 includes a speech perception module 904, as shown in the pipeline of FIG. 9. As discussed below, the speech perception module 904 is configured to divide the voice recording 902 into audio subsegments corresponding to respectively pronounced words 906, resample 908 the audio subsegments to a target sampling audio frequency to generate resampled audio subsegments, perform a Mel transformation 910 to calculate a Mel Frequency Cepstral Coefficients (MFCC) matrix for each of the resampled audio subsegments, and perform feature generation 912 to generate a speech feature vector. The processing pipeline in FIG. 9 also includes a classification module 914 to determine a presence of slurred speech by the person based on the speech feature vector. The classification module 914 outputs a probability of slurred speech 916, which may indicate a stroke.

In some embodiments, slurred speech may be a symptom assessed by the FAST protocol. The subject may be asked to read aloud several standard words in order for their speech to be assessed. It may be assumed that a voice recording 902 of this process is available. The recording itself may be made independently or during the video capturing phase disclosed herein.

In some embodiments, words are shown to the test subject in a timed fashion during the voice recording such that the recording may be automatically split into multiple segments, with each one corresponding to a single one of the words 906. As a result, each test subject voice recording 902 may be transformed into W₁, W₂, . . . , W_{S} audio subsegments corresponding to each pronounced word where S is the number of words shown to the test subject.

In some embodiments, the speech perception module 904 processes each word audio segment Wᵢ individually to resample 908 it to a target sampling audio frequency

$${f}_{sample}^{speech}$$

and then applying the Mel transformation 910 to it in order to calculate the Mel frequency cepstral coefficients (MFCC). As a result, for each word Wᵢ an MFCC matrix MFCCᵢ may be calculated that has a size of

$${N}_{MFCC} \times {N}_{i}^{speech} ,$$

where N_{MFCC} is the target number of cepstral coefficients and

$${N}_{i}^{speech}$$

is the number of audio sample points within Wᵢ. Given the different duration of each word, the feature generation 912 may include constructing a fixed length feature vector

$${x}_{i}^{speech}$$

∈ [symbol] 912 by calculating the first two statistical moments, for example, the mean and the variance of each cepstral coefficient across time, and concatenating them together into a single vector.

In some embodiments, the classification module 914 evaluates whether speech slur is present or not. To do so, the classification module 914 may use a classifier fₛₗᵤᵣ that takes as an input

$${x}_{i}^{speech}$$

and outputs.

$${\hat{y}}_{i}^{speech} = p ( {y}_{slur} = 1 \vert x = {x}_{i}^{speech} ) = {f}_{weakness} ( {x}_{i}^{arm} ) ,$$

where [symbol] ₛₗᵤᵣ∈{0, 1} indicates the presence of slurred speech. After extensive model comparison, the inventors of the present application determined that a Ridge Regression (RR) is well suited for this classification task. Processing the words may result in S predictions

$${\hat{y}}_{1 , ⋯ , N}^{speech} ,$$

which are aggregated using Kernel Density Estimation (KDE) to determine the mean predicted probability of slurred speech 916 as well as the uncertainty of the estimate.

### Detecting Stroke

Certain embodiments merge the predictions of each of the data modalities (e.g., facial asymmetry, arm weakness, and/or slurred speech) by weighing them according to a clinician's expertise as well as by learning from data. Another classifier fₛₜᵣₒₖₑ may be used that takes as an input the predictions made by f_{asymmetry}, f_{weakness} fₛₗᵤᵣ and outputs p(yₛₜᵣₒₖₑ=1). After extensive model comparison, the inventors of the present application determined that a fully connected neural network with two layers is well suited for this classification task.

In some examples, the model disclosed herein is a fully connected neural network with two hidden layers with 100 neurons at each layer and rectified linear unit (ReLU) activation. In certain such examples the ReLU activation is a threshold function that returns the input value if it is positive or zero, and returns zero for any negative input. Mathematically, it may introduce a non-linearity to the neural network model, which enables the network to learn complex patterns and make non-linear transformations.

In some examples, the model may be based on supervised learning wherein labels are provided from a neurological examination. The models disclosed herein, for the disclosed modalities (including stroke prediction), are binary classification models. Thus the models use, for example, the binary entropy loss function as a loss function. The classifiers for each of the modalities (face, arm, speech) may be trained individually and the stroke classifier may be trained separately on output of the other three, classifiers

In some embodiments disclosed herein, probabilities produced by the classifiers may be viewed as a threshold to produce a yes or no answer. The probability may not have to be calibrated to be utilized and may be utilized as a binary output. For example, a produced probably, by a classifier, may result in a yes or no answer.

### Example Experimental Results

Certain embodiments disclosed herein have been tested using data collected from X number of patients that have been split for each of the proposed modalities into the subsets shown in Table 1.

TABLE 1
Patient Conditions and Modalities

| | | | Facial | |
|---|---|---|---|---|
| | Slurred Speech | Arm Weakness | Asymmetry | Stroke |
| Healthy | 36 | 93 | 28 | 25 |
| Affected | 105 | 124 | 122 | 54 |
| Total | 141 | 217 | 150 | 79 |

For every patient, both test data including video, arm motion and speech as well as neurological examination data were collected to provide the ground truth for a training procedure. The models f_{asymmetry}, f_{weakness} fₛₗᵤᵣ, fₛₜᵣₒₖₑ were evaluated by running k-fold validation where 30% of the data was used for evaluation and 70% for training. The average results from the cross validation procedure are summarized in Table 2, while the best obtained model performance is shown in Table 3.

TABLE 2
Average model performance from cross
validation with 100 data splits.

| | Slurred | Arm | Facial | |
|---|---|---|---|---|
| | Speech | Weakness | Asymmetry | Stroke |
| Sensitivity | 0.75 | 0.78 | 0.92 | 0.99 |
| | [95% CI | [95% CI (0.77- | [95% CI (0.92- | [95% CI |
| | (0.74-0.76)] | 0.80)] | 0.92)] | (0.95-1.00)] |
| Specificity | 0.75 | 0.79 | 0.78 | 0.90 |
| | [95% CI | [95% CI (0.77- | [95% CI (0.77- | [95% CI |
| | (0.74-0.76)] | 0.81)] | 0.78)] | (0.80-1.00)] |

TABLE 3
Best model performance from cross validation with 100 data splits.

| | Slurred | Arm | Facial | |
|---|---|---|---|---|
| | Speech | Weakness | Asymmetry | Stroke |
| Sensitivity | 0.93 | 0.82 | 1.00 | 1.00 |
| | [95% CI | [95% CI | [95% CI | [95% CI |
| | (0.78-0.99)] | (0.64-0.93)] | (0.9-1.00)] | (0.84-1.00)] |
| Specificity | 0.90 | 1.00 | 0.88 | 1.00 |
| | [95% CI | [95% CI | [95% CI | [95% CI |
| | (0.56-1.00)] | (0.63-1.00)] | (0.47-1.00)] | (0.69-1.00)] |

Expanding from FAST to BE FAST

Certain embodiments increase the sensitivity and specificity of acute stroke diagnosis by detecting balance abnormalities and/or eye (gaze) abnormalities. For example, the sensors discussed herein may be used to detect balance abnormalities associated with stroke by identifying truncal and appendicular ataxia. The truncal (postural) ataxia can be detected via passive monitoring of accelerometer data. Appendicular (limb) ataxia can be detected from active arm movements, as detailed herein. Example signal patterns an unsteady or tremulous arm associated with imbalance are shown in FIG. 8B and FIG. 8C.

Further, the video processing discussed herein may also be used to track a subject's eyes for abnormalities in gaze movements. For example, a gaze tracking component may detect partial and sustained gaze deviation.

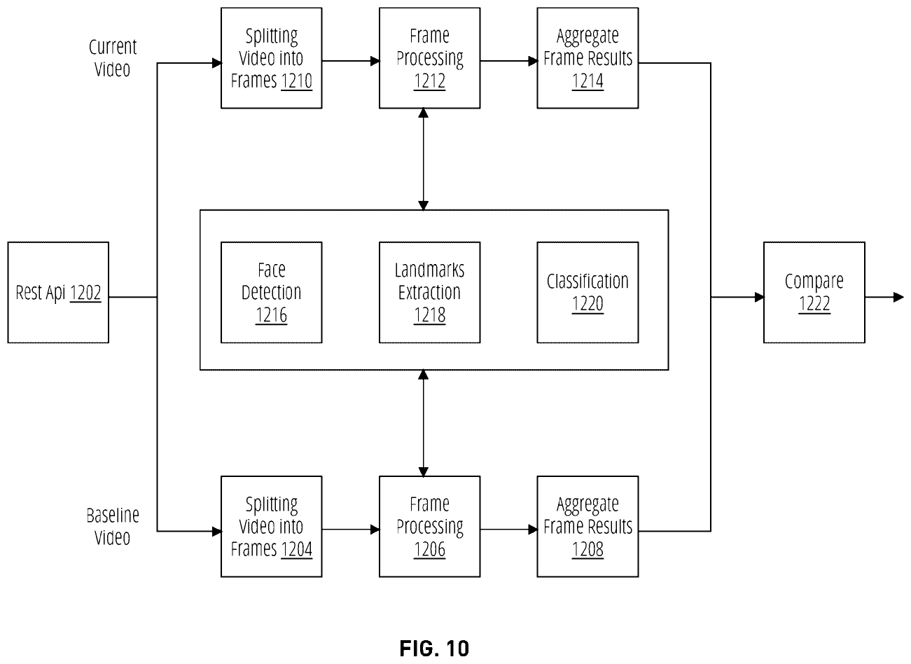

FIG. 10 illustrates an example of a FAST AI online inference pipeline wherein a current video and baseline video may be compared against each other according to one embodiment. The representational state transfer application programming interface (rest api 1202) may provide two videos pipelines, one for a baseline video and one for a current video. The current video may be split into frames 1210. Each frame may then be processed 1212 to, for example, detect a face 1216, extract landmark points 1218, and classify features 1220. The frame results of the current video may then be aggregated 1214 together. The baseline video may be split into frames 1204. Each frame may then be processed 1206 to, for example, detect a face 1216, extract landmark points 1218, and classify features 1220. The frame results of the baseline video may then be aggregated 1214 together. The aggregated video results of the current video 1214 may be compared 1222 to the aggregated video results of the baseline video 1208 to analyze differences thus possibly detecting an occurrence of a stroke.

In some examples, a rest api 1202 may be a set of rules and conventions that allow different software applications to communicate and interact with each other over the internet. It may be based on the principles of the REST architectural style, which emphasizes a stateless, client-server communication mode. API endpoints may provide a standardized way for clients to access and manipulate the resources offered by the server. By following the principles of REST, such as statelessness, uniform interface, and scalability, REST APIs may provide a flexible and scalable approach to building web services that can be easily consumed by various clients, including web browsers, mobile applications, and other software systems.

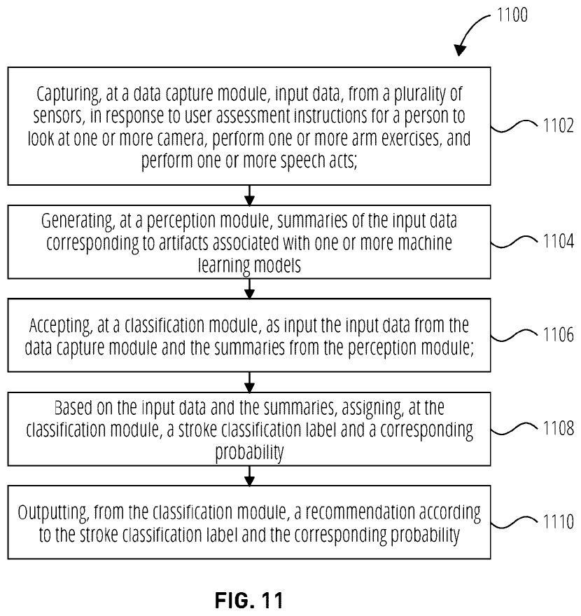

FIG. 11 illustrates a flowchart of a method 1100 for stroke detection, according to embodiments herein. The illustrated method 1100 includes capturing 1102, at a data capture module, input data, from a plurality of sensors, in response to user assessment instructions for a person to look at one or more camera, perform one or more arm exercises, and perform one or more speech acts. The method 1100 further includes generating 1104, at a perception module, summaries of the input data corresponding to artifacts associated with one or more machine learning models. The method 1100 further includes accepting 1106, at a classification module, as input the input data from the data capture module and the summaries from the perception module. The method 1100 further includes, based on the input data and the summaries, assigning 1108, at the classification module, a stroke classification label and a corresponding probability. The method 1100 further includes outputting 1110, from the classification module, a recommendation according to the stroke classification label and the corresponding probability.

In some embodiments, the method 1100 further comprises an instruction module for providing the user assessment instructions for the person who is experiencing a stroke, suspected of experiencing the stroke, or has experienced the stroke. In some such embodiments, the instruction module further instructs the person to sequentially look at the one or more camera, perform the one or more arm exercises, and perform the one or more speech acts. In other embodiments, the instruction module further instructs the person to perform two or more of the user assessment instructions in parallel. In certain embodiments, the instruction module outputs the user assessment instructions as text for a user to read or as synthesized speech.

In some embodiments, the method 1100 further comprises receiving, at the data capture module, the input data from the one or more camera positioned to capture video of a face of the person, and one or more audio capture device configured to record a voice of the person. In some such embodiments, the one or more camera provides at least one of color video and depth data, and the one or more camera may generate arm data corresponding to the one or more arm exercises. In some such embodiments, the data capture module further receives the input data from one or more motion sensor comprising at least one of an accelerometer, a gyroscope, and a magnetometer. The one or more motion sensor may generate arm data corresponding to the one or more arm exercises.

In some embodiments of the method 1100, the artifacts comprise one or more of a pose of a face, location points for the face, a facial asymmetry, a unilateral change of facial movement, an acceleration profile of an arm, an angular velocity of the arm, a speech summary comprising MFCC, a balance profile, and a gaze profile.

In some embodiments of the method 1100, the perception module comprises a face perception module for summarizing captured visual data and depth data from the one or more camera to define a position, a size, and an orientation of a face of the person along with locations of facial landmarks. In some such embodiments, the face perception module includes: a face detector for outputting bounding boxes corresponding to a largest detected face in a sequence of video frames: a facial landmark detector for processing video data corresponding to the bounding boxes to determine the locations of the facial landmarks; and a feature generator for determining a set of facial feature vectors from the facial landmarks for each of the sequence of video frames. In certain such embodiments, the facial landmarks are selected from a group comprising a left eye, a right eye, a left eyebrow, a right eyebrow, a forehead oval, a nose midline, a nose horizontal line, a right NLF, a left NLF, a right cheek, a left cheek, a lip inner circle, and a lip outer circle. Certain such embodiments further comprise using at least 90 location points to define the facial landmarks. In certain such embodiments, the classification module comprises a facial asymmetry submodule for determining a presence of facial asymmetry based on the set of facial feature vectors. In certain such embodiments, the facial asymmetry submodule uses a LDA model to determine the presence of the facial asymmetry. In certain such embodiments, the classification module further comprises a lateral analysis submodule for: measuring movement of a left side of the face of the person and a right side of the face of the person over a period of time: determining an affected side of the face as one of the left side of the face or the right side of the face has less movement over the period of time; and associating the affected side with the presence of the facial asymmetry. In certain such embodiments, for at least one of the face facial asymmetry submodule and a lateral analysis submodule, inference is performed using subsets of the sequence of video frames using a recurrent neural network or using a transformer or attention based architecture.

In certain embodiments of the method 1100, the face perception module accepts as input a video V that is split into frames F_{1, . . . , N}. Each frame Fᵢ may then processed by the face detector that outputs bounding boxes b_{1, . . . , M}_{ᵢ}, where Mᵢ is the number of faces detected in frame Fᵢ. The largest detected face in a frame may be found by applying non-maximal suppression based on the bounding box area such that Bᵢ=argmax({area(bᵢ)|bᵢ∈b_{1, . . . , M}_{ᵢ}}). As a result, there may be N bounding boxes denoted as B_{1, . . . , N}. Each bounding box is then passed through the facial landmark detector 314 resulting in a set Lᵢ={l_{i,1}, i_{i,2}, . . . , l_{i,K}} l_{i,j}∈[0; 1]² is a 2D location with normalized coordinates between 0 and 1 with respect to Bᵢ and K is the number of detected facial landmark points in frame A.

In some such embodiments, the facial landmark detector may be trained to extract a standard 68 key points that are widely used by the machine learning community. See, for example, Hohman. Marc H., et al. “Determining the threshold for asymmetry detection in facial expressions.” The Laryngoscope 124.4 (2014): 860-865. In other embodiments, however, the facial landmark detector 314 may be trained on a custom set of facial landmark points that has been identified by stroke specialists. The features generator may be configured to determine a set of facial feature vectors from the facial landmarks for each of the sequence of video frames. In some cases, directly processing the coordinates of the detected landmark points may yield a classifier with poor generalization capabilities as it may be sensitive to the location and orientation of the face in the image. To reduce or avoid these issues, the facial landmark points may be converted into a set of distances Dᵢ={L₂(l_{i,j}, i_{i,k})|l_{i, j}, l_{i, k}∈Lᵢ, i≠j} with cardinality

$$\vert {D}_{i} \vert = \frac{K * ( K - 1 )}{2}$$

and then a dimensionality reduction may be performed using PCA to obtain a final feature vector

$${x}_{i}^{face}$$

∈ [symbol] for every video frame Fᵢ, where q is the target dimensionality for the PCA step. In some example embodiments, q=100 may be sufficient to explain more than 99% of the variance in Dᵢ for K=68.

In some such embodiments, the classification module, which may include or may be referred to as a facial asymmetry submodule, determines a presence of facial asymmetry based on the set of facial feature vectors. To do so, the classification module may use a classifier f_{asymmetry} that takes as an input

$${x}_{i}^{face}$$

and outputs

$${\hat{y}}_{i}^{face} = p ( {y}_{asymmetry} = 1 \vert x = {x}_{i}^{face} ) = {f}_{asymmetry} ( {x}_{i}^{face} ) ,$$

where y_{asymmetry} ∈{0, 1} may indicate the presence of facial asymmetry. After extensive model comparison, the inventors of the present application determined that a LDA is well suited for this classification task. Processing every frame Fᵢ, i∈{1, . . . , N} in the video may result in N predictions

$${\hat{y}}_{1 , ⋯ , N}^{face}$$

that may be aggregated using a KDE to determine a mean predicted probability of asymmetry as well as an uncertainty of the estimate. In addition, certain embodiments include a lateral analysis submodule to perform a lateral analysis of observed face movements to identify which side of the face is likely affected. The analysis may be based on measuring the total movement of the left and right sides of the face and determining which side has moved less throughout the observed video. In particular, the set of normalized facial landmark points Lᵢ={l_{i,1}, i_{i,2}, . . . , l_{i,K}} may be split into subsets books and now including the facial landmark points that belong to the left and right side of the face, respectively, detected at video frame Fᵢ. Any points along the central vertical line of the face are included in both sets. The total displacement of facial landmark points on each side of the face may be estimated as

$${d}_{i , left} = {\sum }_{{l}_{i , j} ∈ {L}_{i , right}} {L}_{2} ( {l}_{i , j} , {e}_{i , right} ) ,$$

where e_{i, left} and e_{i, right} denote the locations of the center of the left eye and the right eye, respectively, in frame Fᵢ, and L₂ denotes the Euclidean norm. Processing the sequence of video frames results in the sequences d_{1,left}, d_{2,left}, . . . , d_{N,left} and d_{1,right}, d_{2,right}, . . . , d_{N, right} whose variances σ²_{left} and σ²_{right} indicate how much the left and right side of the face has moved throughout the video. The side with the lower variance is predicted to be the affected side.

In some embodiments of the method 1100, the perception module comprises an arm perception module for: resampling multi-dimensional acceleration data, multi-dimensional angular velocity data, and multi-dimensional magnetic field direction data to generate resampled signals comprising an equal sampling frequency and an equal length: truncating the resampled signals to generate truncated signals by removing transitionary artifacts during at least one of a beginning of a test and an end of the test; normalizing magnitudes of the truncated signals to generate normalized signals to account for at least one of different grasps and different sensor orientations: filtering the normalized signals to generate filtered signals by removing noise; and aggregating the filtered signals into an arm motion feature vector. In some such embodiments, the classification module further determines a presence of arm weakness in one of a left arm or a right arm of the person based on the arm motion feature vector. Certain such embodiments, further comprise using, at the classification module, a LR model to determine the presence of the arm weakness.

In some embodiments of the method 1100, the perception module comprises a speech perception module for: dividing a voice recording into audio subsegments corresponding to respectively pronounced words by the person: resampling the audio subsegments to a target sampling audio frequency to generate resampled audio subsegments: applying a Mel transformation to calculate a MFCC matrix for each of the resampled audio subsegments; and processing and concatenate each MFCC matrix to generate a speech feature vector. In some such embodiments, the classification module determines a presence of slurred speech by the person based on the speech feature vector. In certain such embodiments, the classification module uses an RR model to determine the presence of the slurred speech.

In some embodiments of the method 1100, the classification module merges predictions of facial asymmetry, arm weakness, and slurred speech to determine the stroke classification label as healthy or affected and the corresponding probability based on a connected neural network model with two layers. In some such embodiments, the classification module further comprises merging predictions of one or more of truncal ataxia, appendicular ataxia, and gaze tracking to determine the stroke classification label and the corresponding probability.

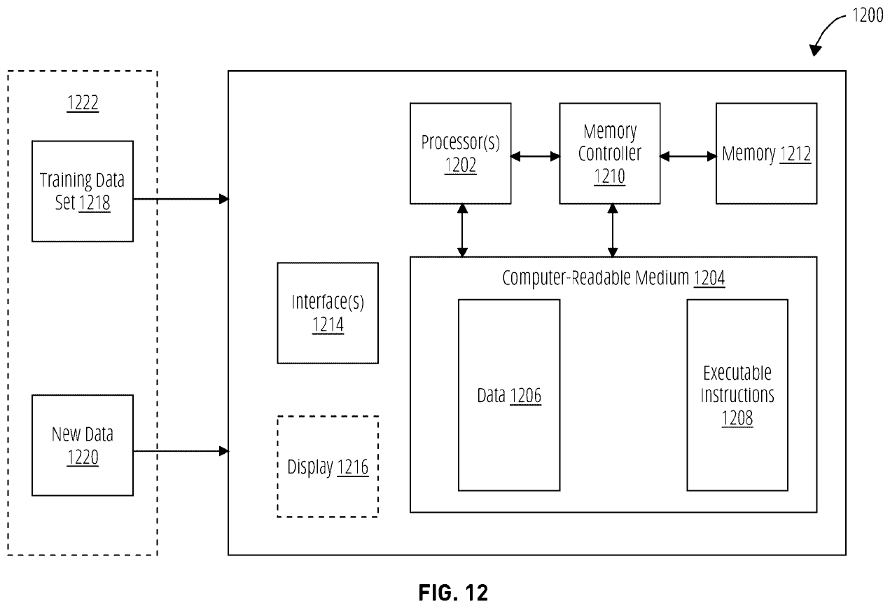

FIG. 12 is a schematic illustration of a computing system arranged in accordance with examples of the present disclosure. The computing system 1200 may be used to implement one or more machine learning models, such as the machine learning models described in FIG. 1 to FIG. 10.

The computer-readable medium 1204 may be accessible to the processor(s) 1202. The computer-readable medium 1204 may be encoded with executable instructions 1208. The executable instructions 1208 may include executable instructions for implementing a machine learning model to, for example, stroke detection. The executable instructions 1208 may be executed by the processor(s) 1202. In some examples, the Executable instructions 1208 may also include instructions for generating or processing training data sets and/or training a machine learning model. Alternatively or additionally, in some examples, the machine learning model, or a portion thereof, may be implemented in hardware included with the computer-readable medium 1204 and/or processor(s) 1202, for example, application-specific integrated circuits (ASICs) and/or field programmable gate arrays (FPGA).

The computer-readable medium 1204 may store data 1206. In some examples, the data 1206 may include one or more training data sets, such as training data set 1218. The training data may be based on a selected application. For example, the training data set 1218 may include one or more sequences of images, one or more audio files, and/or one or more motion data files. In some examples, training data set 1218 may be received from another computing system (e.g., a data acquisition module 1222, a cloud computing system). In other examples, the training data set 1218 may be generated by the computing system 1200. In some examples, the training data sets may be used to train one or more machine learning models. In some examples, the data 1206 may include data used in a machine learning model (e.g., weights, connections between nodes). In some examples, the data 1206 may include other data, such as new data 1220. The new data 1220 may include one or more image sequences, audio files, and/or motion data files not included in the training data set 1218. In some examples, the new data may be analyzed by a trained machine learning model to detect a stroke. In some examples, the data 1206 may include outputs, as described herein, generated by one or more machine learning models implemented by the computing system 1200. The computer-readable medium 1204 may be implemented using any medium, including non-transitory computer readable media. Examples include memory, random access memory (RAM), read only memory (ROM), volatile or non-volatile memory, hard drive, solid state drives, or other storage. While a single medium is shown in FIG. 12, multiple media may be used to implement computer-readable medium 1204.

In some examples, the processor(s) 1202 may be implemented using one or more central processing units (CPUs), graphical processing units (GPUs), ASICS, FPGAs, or other processor circuitry. In some examples, the processor(s) 1202 may execute some or all of the executable instructions 1208. In some examples, the processor(s) 1202 may be in communication with a memory 1212 via a memory controller 1210. In some examples, the memory 1212 may be volatile memory, such as dynamic random-access memory (DRAM). The memory 1212 may provide information to and/or receive information from the processor(s) 1202 and/or computer-readable medium 1204 via the memory controller 1210 in some examples. While a single memory 1212 and a single memory controller 1210 are shown, any number may be used. In some examples, the memory controller 1210 may be integrated with the processor(s) 1202.

In some examples, the interface(s) 1214 may provide a communication interface to another device (e.g., the data acquisition module 1222), a user, and/or a network (e.g., LAN, WAN, Internet). The interface(s) 1214 may be implemented using a wired and/or wireless interface (e.g., Wi-Fi, BlueTooth, HDMI, USB, etc.). In some examples, the interface(s) 1214 may include user interface components which may receive inputs from a use. Examples of user interface components include a keyboard, a mouse, a touch pad, a touch screen, and a microphone. In some examples, the interface(s) 1214 may communicate information, which may include user inputs, data 1206, training data set 1218, and/or new data 1220, between external devices (e.g., the data acquisition module 1222) and one or more components of the computing system 1200 (e.g., processor(s) 1202 and computer-readable medium 1204).

In some examples, the computing system 1200 may be in communication with a display 1216 that is a separate component (e.g., using a wired and/or wireless connection) or the display 1216 may be integrated with the computing system. In some examples, the display 1216 may display data 1206 such as outputs generated by one or more machine learning models implemented by the computing system 1200. Any number or variety of displays may be present, including one or more LED, LCD, plasma, or other display devices.

In some examples, the training data set 1218 and/or new data 1220 may be provided to the computing system 1200 via the interface(s) 1214. Optionally, in some examples, some or all of the training data set 1218 and/or new data 1220 may be provided to the computing system 1200 by one or more sensors of the data acquisition module 1222, such as the data acquisition module data acquisition devices 104 shown in FIG. 1 or the data acquisition module 206 shown in FIG. 2. In some examples, the data acquisition module 1222 may include a color camera or video camera, an audio capture device, motion sensors (e.g., accelerometers), or a combination thereof.

For one or more embodiments, at least one of the components set forth in one or more of the preceding figures may be configured to perform one or more operations, techniques, processes, and/or methods as set forth herein. For example, a processor as described herein in connection with one or more of the preceding figures may be configured to operate in accordance with one or more of the examples set forth herein.

Any of the above described embodiments may be combined with any other embodiment (or combination of embodiments), unless explicitly stated otherwise. The foregoing description of one or more implementations provides illustration and description, but is not intended to be exhaustive or to limit the scope of embodiments to the precise form disclosed. Modifications and variations are possible in light of the above teachings or may be acquired from practice of various embodiments.

Embodiments and implementations of the systems and methods described herein may include various operations, which may be embodied in machine-executable instructions to be executed by a computer system. A computer system may include one or more general-purpose or special-purpose computers (or other electronic devices). The computer system may include hardware components that include specific logic for performing the operations or may include a combination of hardware, software, and/or firmware.

It should be recognized that the systems described herein include descriptions of specific embodiments. These embodiments can be combined into single systems, partially combined into other systems, split into multiple systems or divided or combined in other ways. In addition, it is contemplated that parameters, attributes, aspects, etc. of one embodiment can be used in another embodiment. The parameters, attributes, aspects, etc. are merely described in one or more embodiments for clarity, and it is recognized that the parameters, attributes, aspects, etc. can be combined with or substituted for parameters, attributes, aspects, etc. of another embodiment unless specifically disclaimed herein.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the description is not to be limited to the details given herein, but may be modified within the scope and equivalents of the appended claims.

1. A stroke detection system comprising:
one or more processors; and
a memory storing executable instructions that, when executed by the one or more processors, implement:
a data capture module to capture input data from a plurality of sensors, in response to user assessment instructions for a person to look at one or more camera, perform one or more arm exercises, and perform one or more speech acts;
a perception module to generate summaries of the input data corresponding to artifacts associated with one or more machine learning models; and
a classification module to:
accept as input the input data from the data capture module and the summaries from the perception module:
based on the input data and the summaries, assign a stroke classification label and a corresponding probability; and
output a recommendation according to the stroke classification label and the corresponding probability.

2. The stroke detection system of claim 1, wherein the executable instructions, when executed by the one or more processors, further implement an instruction module to provide the user assessment instructions for the person who is experiencing a stroke, suspected of experiencing the stroke, or has experienced the stroke.

3. The stroke detection system of claim 2, wherein the instruction module is further to instruct the person to sequentially look at the one or more camera, perform the one or more arm exercises, and perform the one or more speech acts.

4. The stroke detection system of claim 2, wherein the instruction module is further to instruct the person to perform two or more of the user assessment instructions in parallel.

5. The stroke detection system of claim 2, wherein the instruction module outputs the user assessment instructions as text for a user to read or as synthesized speech.

6. The stroke detection system of claim 1, wherein the data capture module receives the input data from the one or more camera positioned to capture video of a face of the person, and one or more audio capture device configured to record a voice of the person.

7. The stroke detection system of claim 6, wherein the one or more camera is configured to provide at least one of color video and depth data, and wherein the one or more camera is configured to generate arm data corresponding to the one or more arm exercises.

8. The stroke detection system of claim 6, wherein the data capture module further receives the input data from one or more motion sensor comprising at least one of an accelerometer, a gyroscope, and a magnetometer, the one or more motion sensor to generate arm data corresponding to the one or more arm exercises.

9. The stroke detection system of claim 1, wherein the artifacts comprise one or more of a pose of a face, location points for the face, a facial asymmetry, a unilateral change of facial movement, an acceleration profile of an arm, an angular velocity of the arm, a speech summary comprising Mel Frequency Cepstral Coefficients (MFCC), a balance profile, and a gaze profile.

10. The stroke detection system of claim 1, wherein the perception module comprises a face perception module configured to summarize captured visual data and depth data from the one or more camera to define a position, a size, and an orientation of a face of the person along with locations of facial landmarks.

11. The stroke detection system of claim 10, wherein the face perception module comprises:
a face detector that outputs bounding boxes corresponding to a largest detected face in a sequence of video frames;
a facial landmark detector that processes video data corresponding to the bounding boxes to determine the locations of the facial landmarks; and
a feature generator to determine a set of facial feature vectors from the facial landmarks for each of the sequence of video frames.

12. The stroke detection system of claim 11, wherein the facial landmarks are selected from a group comprising a left eye, a right eye, a left eyebrow, a right eyebrow, a forehead oval, a nose midline, a nose horizontal line, a right nasolabial fold (NLF), a left NLF, a right cheek, a left cheek, a lip inner circle, and a lip outer circle.

13. The stroke detection system of claim 12, wherein at least 90 location points are used to define the facial landmarks.

14. The stroke detection system of claim 11, wherein the classification module comprises a facial asymmetry submodule to determine a presence of facial asymmetry based on the set of facial feature vectors.

15. The stroke detection system of claim 14, wherein the facial asymmetry submodule uses a Linear Discriminant Analysis (LDA) model to determine the presence of the facial asymmetry.

16. The stroke detection system of claim 14, wherein the classification module further comprises a lateral analysis submodule to:
measure movement of a left side of the face of the person and a right side of the face of the person over a period of time;
determine an affected side of the face as one of the left side of the face or the right side of the face has less movement over the period of time; and
associate the affected side with the presence of the facial asymmetry.

17. The stroke detection system of claim 14, wherein for at least one of the face facial asymmetry submodule and a lateral analysis submodule, inference is performed using subsets of the sequence of video frames using a recurrent neural network or using a transformer or attention based architecture.

18. The stroke detection system of claim 1, wherein the perception module comprises an arm perception module to:
resample multi-dimensional acceleration data, multi-dimensional angular velocity data, and multi-dimensional magnetic field direction data to generate resampled signals comprising an equal sampling frequency and an equal length;
truncate the resampled signals to generate truncated signals by removing transitionary artifacts during at least one of a beginning of a test and an end of the test;
normalize magnitudes of the truncated signals to generate normalized signals to account for at least one of different grasps and different sensor orientations;
filter the normalized signals to generate filtered signals by removing noise; and
aggregate the filtered signals into an arm motion feature vector.

19. The stroke detection system of claim 18, wherein the classification module is configured to determine a presence of arm weakness in one of a left arm or a right arm of the person based on the arm motion feature vector.

20. The stroke detection system of claim 19, wherein the classification module uses a Logistic Regression (LR) model to determine the presence of the arm weakness.

21. The stroke detection system of claim 1, wherein the perception module comprises a speech perception module to:
divide a voice recording into audio subsegments corresponding to respectively pronounced words by the person;
resample the audio subsegments to a target sampling audio frequency to generate resampled audio subsegments;
apply a Mel transformation to calculate a Mel Frequency Cepstral Coefficients (MFCC) matrix for each of the resampled audio subsegments; and
process and concatenate each MFCC matrix to generate a speech feature vector.

22. The stroke detection system of claim 21, wherein the classification module is configured to determine a presence of slurred speech by the person based on the speech feature vector.

23. The stroke detection system of claim 22, wherein the classification module uses a Ridge Regression (RR) model to determine the presence of the slurred speech.

24. The stroke detection system of claim 1, wherein the classification module merges predictions of facial asymmetry, arm weakness, and slurred speech to determine the stroke classification label as healthy or affected and the corresponding probability based on a connected neural network model with two layers.

25. The stroke detection system of claim 24, wherein the classification module further merges predictions of one or more of truncal ataxia, appendicular ataxia, and gaze tracking to determine the stroke classification label and the corresponding probability.

26-50. (canceled)
